(19) United States
(12) Patent Application Publication
(10) Pub. No.: US 2025/0319248 A1
(43) Pub. Date: Oct. 16, 2025

(54) TUBE ANTI-EXTRACTION DEVICE

(71) Applicants: PAX CO., LTD., Tokyo (JP); MEDIKIT CO., LTD., Tokyo (JP)

(72) Inventors: Yutaka SUZUKI, Tokyo (JP); Takahiro SHIBATA, Tokyo (JP)

(21) Appl. No.: 19/172,108

(22) Filed: Apr. 7, 2025

(30) Foreign Application Priority Data
Apr. 11, 2024 (JP) 2024-63879
Mar. 31, 2025 (JP) 2025-58856

(51) Int. Cl. A61M 5/158

(52) U.S. Cl. CPC A61M 5/158; A61M 2005/1586

(57) ABSTRACT

A tube anti-extraction device includes a case, a resistance member provided within the case, and a tool for attaching the case near a puncture site. The case includes openable and closable upper and lower half bodies, and a removal-preventing or retaining column is erected inside. A tube that has entered the case through the hole used as an inlet of the case is wound around the column one and a half turns, and exits to the outside through the hole used as an outlet. After the tube is attached, the upper and lower half bodies are closed and coupled by an engaging portion. Even when the tube is extracted from the outside, the tube wound around the column resists this, and the tube is prevented from being extracted or removed and retained by the device.

### CROSS-REFERENCE TO RELATED APPLICATIONS

This application claims the benefit priority to Japanese Patent Applications No. 2024-63879 filed on Apr. 11, 2024 and No. 2025-58856 filed on Mar. 31, 2025, the contents of which are incorporated herein by reference.

### TECHNICAL FIELD

The present invention relates to an anti-extraction device for peripheral intravenous (IV) tubes (including blood transfusion tubes), a peripherally inserted central catheter (PICC) line, hemodialysis tubes, and the like.

In this specification, the tube includes a catheter, and anti-extraction is a concept including prevention of removal, prevention of withdrawal of the tube, or the like, and therefore retaining, fixing, holding or keeping the tube. The anti-extraction includes preventing in advance the tube from being extracted as a result of carelessness, an accident, or the like, and intentionally or manually extracting, pulling or drawing the tube. When the tube is extracted, a catheter or the like connected to one end of the tube and punctured into an arm or the like of a human body may come out, leading to a serious accident.

### BACKGROUND

An example of a tube removal/kink prevention device for a drip infusion is described in Japanese Patent No. 6656873.

The tube removal/kink prevention device described in Japanese Patent No. 6656873 includes a case that includes an inlet and an outlet and a tube is passed through the inlet and the outlet, a drum that is rotatably incorporated in the case and around which the inserted tube is wound; and an elastic unit that is provided between the case and the drum, allows rotation of the drum and drawing of the tube when a tensile force acts on the tube, and reverses the drum by an elastic return force to draw back the tube when the tensile force is released.

According to the example described in Japanese Patent No. 6656873, this removal/kink prevention device is attached to the IV device. That is, in the IV device, a pole is erected on a base with casters, and the infusion bag is hung on an arm fixed to an upper portion of the pole.

The drip tube is connected to the lower part of the infusion bag from an IV needle (catheter) punctured into the forearm of the patient, through a drum of the removal/kink prevention device fixed to the lower part of the pole of the stand, and further through an infusion pump provided in the stand. The IV device is generally placed adjacent to the bed on which the patient lies.

The drip tube connected to the IV needle punctured in the forearm of the patient has a slight length margin (slack) between the drip tube and the removal/kink prevention device, but if the patient moves the arm more than necessary, the drip tube may be pulled. However, since the drum of the removal/kink prevention device rotates according to the removal of the drip tube, the tube is extracted, and a large load is not applied to the tube and the IV needle.

When the patient returns the arm to the original position, the elastic unit of the removal/kink prevention device reversely rotates the drum and winds the tube, so that the tube returns to the original position.

The IV needle punctured into the arm of the patient is only fixed to the arm with a fixing tape, and this portion is unprotected. A patient in need of an instillation moves together with an IV stand when going to the toilet and when performing rehabilitation. In particular, in rehabilitation, it is required to take various postures and perform various movements for recovery of motor function. At such times, the patient and the trainer must be careful not to apply force to the portion of the needle punctured into the arm. This is because a tensile force may be unintentionally applied to the drip tube.

Non-routine accidents can also occur. An accident in which a patient in a confusion state, a patient with dementia, or a patient in another abnormal mental state pulls the drip tube intentionally or unconsciously and pulls out the IV needle is also assumed. The same accident may occur not only for the drip tube but also for the various tubes described above.

### SUMMARY OF THE INVENTION

An object of the present invention is to provide a device capable of previously preventing removal of a tube and retaining the tube connected to a needle (catheter) punctured into a human body regardless of intention or carelessness, or pulling with a strong force. In addition, an object of the present invention is to prevent extracting of a tube due to carelessness or the like without causing too much discomfort even if the tube is accompanied by a tube such as a drip infusion, and to enable life with medical treatment with less anxiety in a hospital or the like.

The target tubes of the anti-extraction device according to the present invention are roughly divided into a peripheral IV tube (usually, since blood transfusion is performed from a peripheral IV tube, it is included in the IV tube), a peripherally inserted central catheter (PICC) tube (line), and a hemodialysis tube. Peripheral IV tubes usually puncture the forearm or lower extremity. Lower limbs (lower leg and dorsum of the foot) are rarely punctured. Peripherally inserted central catheter (PICC) lines (tubes) usually puncture the inside of the upper arm. Hemodialysis tubes usually puncture the forearm. In the case of indwelling a drip tube or the like in a blood vessel, usually, a vessel is punctured with a device (the whole may be referred to as an IV needle) including a needle (inner tube) with a sharp tip and a synthetic resin catheter (outer tube) with a lumen, after it is confirmed that the drip tube has been indwelled in the blood vessel, the inner tube is pulled out, a synthetic resin catheter is indwelled in the blood vessel, and a tube extending outward is connected thereto.

The tube anti-extraction device according to the present invention includes a case having a sidewall (side surface) formed with a hole for passing a tube therethrough, a resistance member provided within the case for resisting a tensile force acting from outside the case on the tube passing out of the hole, and a tool for attaching the case to the wrist on the upper limb side or the ankle on the lower limb side where the puncture site (area) is located. The upper limb is a generic name collectively referred to as a lower extremity (dorsum of the hand), a forearm (including wrist), and an upper arm, and the lower limb is a generic name of a dorsum of the foot, a lower leg (including ankle), and a thigh. The wrist on the upper limb side where the puncture site is present specifically refers to a wrist of an arm or a lower extremity to which the tip of the tube to be prevented from being extracted is attached. For example, if the tube is attached to the right arm, the wrist is the wrist of the right arm.

The side wall (side surface) of the case includes a peripheral wall surface in a case where the case has a circular shape, an elliptical shape, or the like in a plan view. In one embodiment, two holes through which the tube passes are formed in the side wall (surface) of the case. In another embodiment, four holes through which the tube passes are formed in the side wall (surface) of the case. The number of holes formed on the side wall (surface) of the case may be any number other than the above, such as one, three, or five or more. The tube enters the case from one hole of the case and is led out of the case from the same hole or another hole. The hole is used as an inlet or an outlet for the tube (guide hole).

Even if the tube coming out of the hole of the case is inadvertently, accidentally, or intentionally pulled, the case is attached to the wrist or the ankle by the attaching tool, and the resistance member in the case resists the pulling of the tube, so that the movement of the tube is prevented. In this way, removal of the tube is prevented. In addition, since the case is only attached to the wrist or the ankle, there is little or a little discomfort given to the patient.

In one embodiment, the resistance member is all or a part of a removal-preventing or retaining column around which the tube is wound and a curved or linear groove or hole for preventing removal or retaining the tube along which the tube is running in the case. The removal-preventing or retaining column is preferably formed of a soft member, of for example, soft rubber. The frictional force with the tube can be kept high by the soft member. In order to prevent the tube from falling off, a flange extending outward is preferably provided on the upper portion of the removal-preventing or retaining column. In one embodiment, the removal-preventing or retaining column includes a soft member crown that covers the receiving portion in the case.

Preferably, the case has at least two partial bodies, and these partial bodies are separably connected by one or more combinations of a hinge, an engagement, a fitting, and a screw-fitting. Then, absent portions for hole formation are formed in each of the at least two partial bodies, and when the partial bodies are combined, the absent portions are met to form the hole. Since the case is formed by the coupling of at least two partial bodies, it is convenient to operate since the partial bodies can be separated or opened (uncoupled) and the tube placed there so that the resistance member acts.

Furthermore, for the sake of caution, it is advisable to further includes a fastener or a lock for keeping the at least two partial bodies forming the case coupled.

The present invention also provides a case for the anti-extraction device. This case includes a case body having a side wall formed with a hole for passing a tube therethrough, and a resistance member provided within the case body for resisting a tensile force acting from outside the case body on the tube passing out of the hole.

### BRIEF DESCRIPTION OF THE DRAWINGS

FIG. 1 is a perspective view illustrating a use state of a tube anti-extraction device according to a first embodiment;

FIG. 2 illustrates the inside of a case by opening an upper half body and a lower half body;

FIG. 3 is a cross-sectional view taken along line III-III of FIG. 2;

FIG. 4 is a view as seen from line IV-IV of FIG. 2;

FIG. 5 illustrates a cross section of a wrist in a state where the case is attached to the wrist;

FIG. 6 illustrates a state in which the case is locked;

FIG. 7 is a perspective view illustrating a state in which another lock (padlock) is attached to the case;

FIG. 8 illustrates a second embodiment and illustrates the inside with the upper and lower half bodies of the case open;

FIG. 9 is a cross-sectional view taken along line IX-IX of FIG. 8;

FIG. 10 is a view as seen from line X-X of FIG. 8;

FIG. 11 is a cross-sectional view of the wrist showing the case being worn on the wrist;

FIG. 12 illustrates a third embodiment and illustrates the inside of a lower half body;

FIG. 13 illustrates the third embodiment and illustrates the inside of the lower half body;

FIG. 14 illustrates the third embodiment and illustrates the inside of the lower half body;

FIG. 15 is a plan view illustrating a modified embodiment of the case;

FIG. 16 illustrates a modified embodiment and illustrates the inside with the upper and lower half bodies open;

FIG. 17 is a front view illustrating a modified embodiment of FIG. 16;

FIG. 18 illustrates another modified embodiment and illustrates the inside with the upper and lower half bodies open;

FIG. 19 illustrates a fourth embodiment, and is a plan view illustrating the entire tube anti-extraction device having four holes;

FIG. 20 is a plan view illustrating a case in an open state;

FIG. 21 is a side view of the opened case;

FIG. 22 is a perspective view illustrating how removal-preventing or retaining columns are assembled;

FIG. 23 is an enlarged cross-sectional view illustrating a fastener provided in a case;

FIG. 24 is a plan view illustrating a case of a tube anti-extraction device having two holes;

FIG. 25 illustrates a using method of a device having four holes;

FIG. 26 illustrates another using method of a device having four holes; and

FIG. 27 illustrates a use state of the device of the fourth embodiment.

### DETAILED DESCRIPTION

### First Embodiment

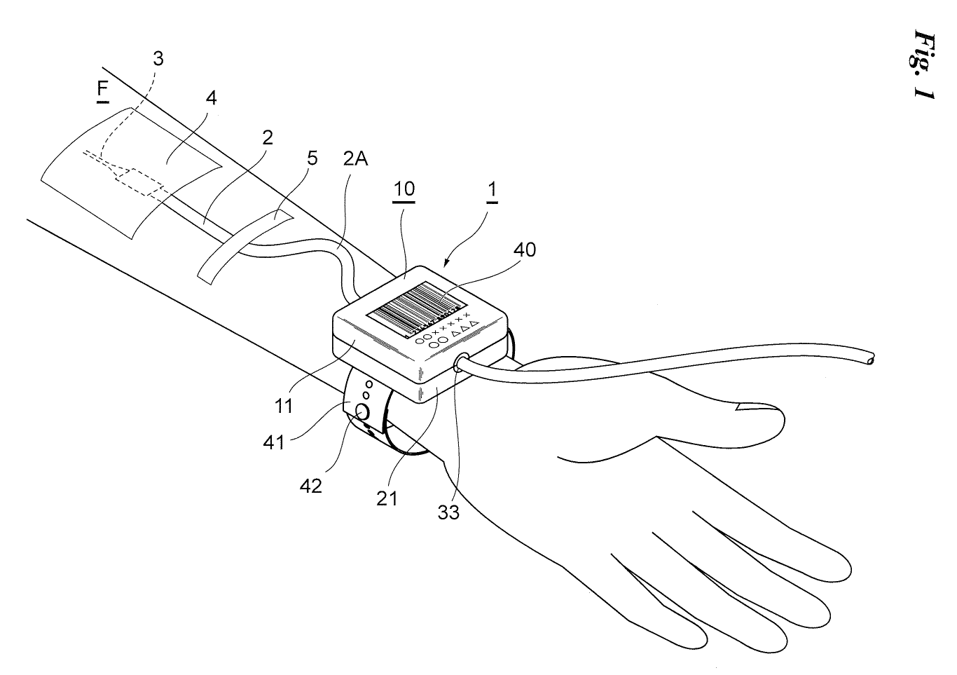

FIG. 1 illustrates a use state of a tube anti-extraction device according to a first embodiment. This embodiment is intended to prevent the tube (catheter) connected to an intravenous (IV) needle from being extracted, removed or pulled.

A tube anti-extraction device 1 includes a case (case body) 10 and a perforated band (attaching tool) 41 (band in which a plurality of holes are formed at intervals) for loosely attaching the case 10 to a wrist of a person. The central portion of the band 41 is strongly adhered to the lower surface of the case 10 or fixed by another method (implantation, penetration, or the like). On the upper surface of the case 10, paper on which a one-dimensional barcode 40 (which may be a two-dimensional code) representing a patient identification code (patient number) and other information is printed is adhered. The case 10 includes two half bodies (partial bodies) (for convenience, and they are referred to as an upper half body 11 and a lower half body 21).

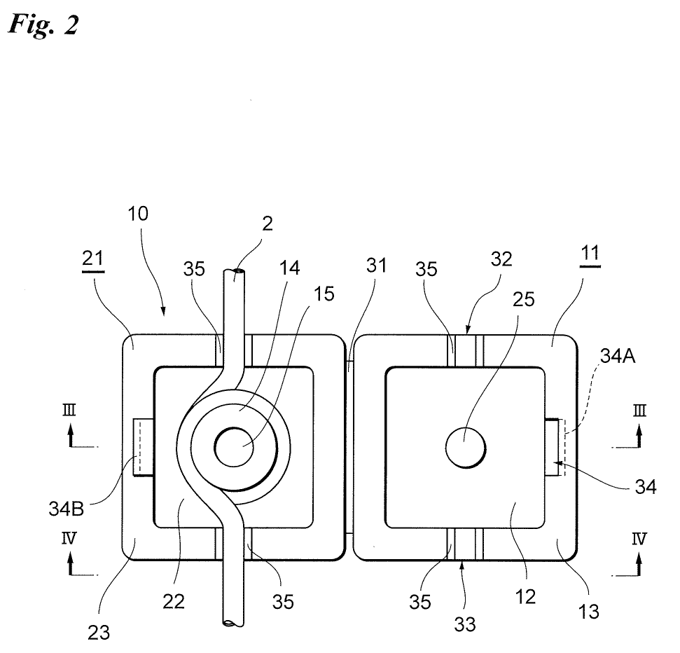

A forearm F is punctured with an IV needle (catheter) 3, and a drip tube 2 connected to the IV needle (catheter) 3 via the connector enters the case 10 from an inlet (hole) 32 (see FIG. 2) of the case 10 of the device 1, exits from the outlet (hole) 33 of the case 10 to the outside of the case 10, and is connected to an infusion bag (not illustrated) suspended on an IV stand (not illustrated). The IV needle (catheter) 3 and the like are protected by a fixing tape 4. On the device 1 side near the fixing tape 4, the drip tube 2 is fixed to the forearm F by a tape 5, and a slight slack (indicated by reference numeral 2A) is secured in the drip tube 2 between the tape 5 and the device 1. As can be seen from FIG. 1, in a case where the forearm F to be punctured for indwelling the drip tube (catheter) is the left arm (upper limb), the device 1 is also attached to the wrist of the left arm.

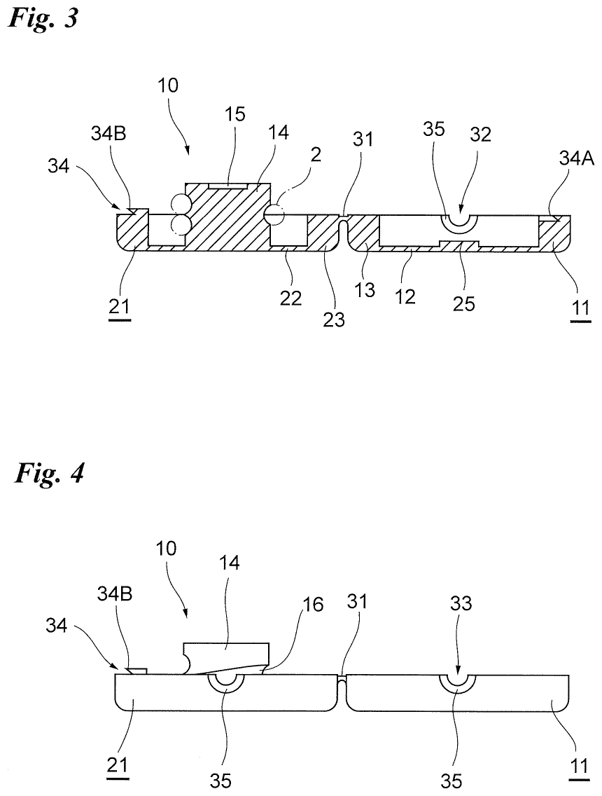

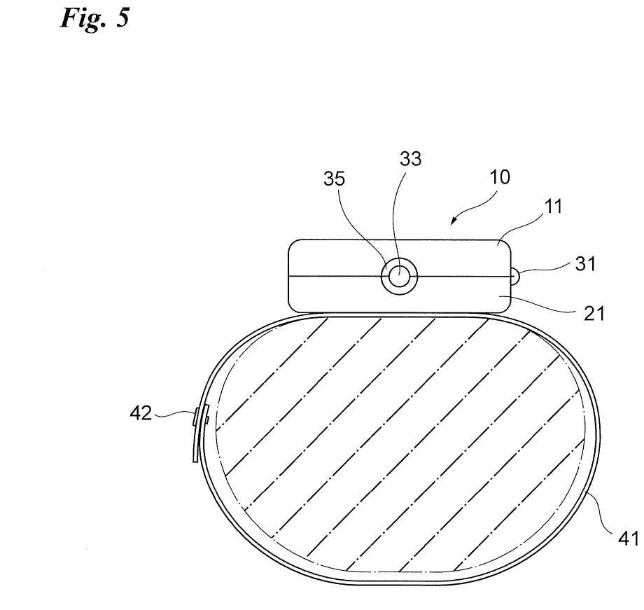

FIG. 2 illustrates a state in which the upper half body 11 and the lower half body 21 of the case 10 are opened, that is, the inside of the case 10. FIG. 3 is a cross-sectional view taken along line III-III of FIG. 2, and FIG. 4 is a view as seen from line IV-IV of FIG. 2. FIG. 5 illustrates a state in which the case 10 is attached to the forearm F near the wrist, in a cross section of the wrist.

With reference to these drawings, the upper half body 11 and the lower half body 21 of the case 10 are openably coupled by a hinge 31. The upper half body 11 includes a bottom surface (bottom plate) (corresponding to the upper surface (upper plate) of the case 10) 12 and a frame portion 13 around the bottom surface. Similarly, the lower half body 21 includes a bottom surface (bottom plate) 22 and a frame portion 23 around the bottom surface. The upper half body 11 and the lower half body 21 are freely openable and closable via the hinge 31, and when they are closed, the case 10 becomes a slightly thin rectangular parallelepiped as a whole (rounded at corner portions). The central portions in the width direction of the facing end surfaces (side surfaces) (side walls) of the frame portions 13 and 23 of the upper and lower half bodies 11 and 21 are absent (lacking) (recess) in a semicircular shape so that an inlet 32 and an outlet 33 for the tube 2 are formed when the upper and lower half bodies 11 and 21 are closed. A resistive half ring (inlet/outlet resistor) 35 that holds (retains) the movement of the tube 2 (applies some pressure) when the upper and lower half bodies 11 and 21 are closed is fixedly fitted inside the semicircular absent portion. These half rings 35 become annular when the upper and lower half bodies 11 and 21 are closed, and tighten the tube 2 from the outside thereof. The half ring 35 is formed of soft rubber, sponge, or the like having high frictional resistance. The inlet and outlet 32 and 33 may hold the tube 2 in the case 10 to the extent that the tube 2 is not loosened. When the lower half body 21 is referred to as a case main body, the upper half body 11 can be referred to as a lid.

A removal-preventing or retaining column (column for winding) (resistance member) (simply referred to as “removal-preventing column” hereinafter) 14 is fixedly erected on and fixed to a bottom surface (bottom plate) 22 of a central portion in the lower half body 21. A recess 15 is formed on the top surface (top plate) of the removal-preventing column 14, and a spiral groove 16 is formed on the peripheral surface of the column 14. On the other hand, on the inner surface of the bottom surface of the upper half body 11, a protrusion 25 that fits into the recess 15 of the column 14 when the upper and lower half bodies 11 and 21 are closed is fixedly formed. It is preferable that the removal-preventing column 14 is formed of rubber having high frictional resistance or the peripheral surface (groove 16) of the removal-preventing column 14 is provided with an anti-slip coating.

Further, in the frame portions 13 and 23 of the upper and lower half bodies 11 and 21, respectively, a fastening groove 34A and a fastening claw 34B that engage (lock) with each other when the upper and lower half bodies 11 and 21 are closed are formed. The fastening groove 34A and the fastening claw 34B constituting an engaging portion 34 (fastener) may be formed in either the upper half or the lower half body. As the fastener, a fitting portion to be fitted to each other may be provided at all or a part of the periphery of the frame portions 13 and 23 instead of the engaging portion 34.

The upper and lower half bodies 11 and 21 of the case 10, including the engaging portion and the like, are preferably integrally formed of synthetic resin.

A perforated band (a plurality of holes are formed at intervals in the length direction) (attaching tool) 41 whose substantially central portion is firmly bonded to the lower surface of the case 10 is wound around the wrist of the patient, appropriate holes at both ends are aligned, and the case 10 is attached to the wrist of the patient by fastening with a hook 42. It is desirable that the hook 42 cannot be easily separated once it is temporarily fastened. The perforated band 41 is slightly loose, but does not come off from the hand. The upper half body 11 (lid) of the case 10 is opened, the drip tube 2 (the IV needle (catheter) 3 has already been punctured and fixed with the fixing tape 4) is wound around the removal-preventing column 14 of the lower half body 21 of the case 10 about one and a half times along the spiral groove 16, and the front and rear thereof are fitted into the semicircular absent portion, and the upper half body 11 is closed. As a result, the tube 2 enters the case 10 from the inlet 32 of the case 10, is wound around the removal-preventing column 14, and goes out from the outlet 33. At this time, a slack 2A is applied between the case 10 and the puncture position of the IV needle (catheter) 3, and the tube 2 is fixed to the forearm F of the patient with the tape 5 near the puncture position. When the upper half body 11 is closed, the upper and lower half bodies 11 and 21 are held in a closed state by the engaging portion 34. Depending on the drip tube, a connector may be provided at a position away from the outlet 33 of the case 10 for attachment and detachment. Even in this case, the tube is restrained by the case in the same manner.

Even if a tensile force acts on the tube 2 that has exited from the outlet 33 of the case 10 to the outside due to carelessness, an accident, or the like, or intentionally, since the tube 2 is wound around the removal-preventing column 14 in the case 10 and is pressed by the inlet 32 and the outlet 33 of the case 10, the tube 2 does not come out of the case 10 by the frictional force (fixed to the case). Since the case 10 is only loosely constrained to the wrist by the band 41, if the tensile force applied to the tube 2 is strong, the case 10 moves in the direction of the hand but does not come out of the hand. Since the movement of the case 10 is absorbed by the slack 2A, a tensile force does not act on the IV needle (catheter) 3.

In this way, the tube will not be removed even if a strong external tensile force is applied intentionally or unintentionally due to carelessness, accident, or the like. In addition, the tube does not come out due to carelessness or the like, the IV needle (catheter) is kept safe, and the patient can concentrate on medical care such as rehabilitation with a sense of security. Since the case 10 is loosely attached to the wrist by the band 41, the influence on the patient is to the extent of wearing a wristwatch, and the patient hardly feels uncomfortable even if the case 10 is attached. It is rare that the slack 2A between the inlet 32 side of the case 10 and the IV needle (catheter) 3 is intentionally pulled. However, when it is desired to prevent this, the slack 2A may be disposed along the surface of the arm of the patient, a synthetic resin film may be provided to cover the slack 2A, and the entire periphery of the synthetic resin film may be attached to the arm of the patient with a tape.

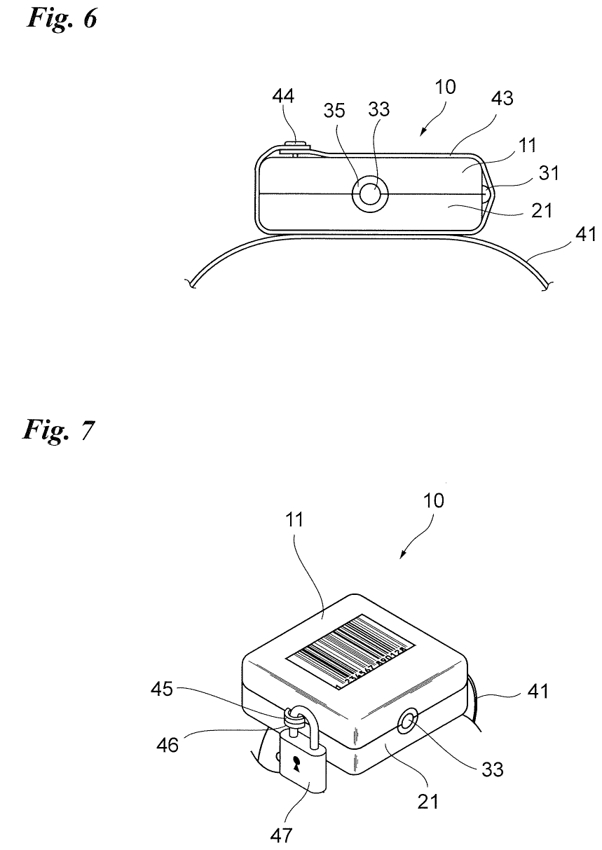

The upper and lower half bodies 11 and 21 of the case 10 are held in a closed state by the engaging portion 34, but may be opened if forcibly opened by a strong force. FIGS. 6 and 7 illustrate an example in which the case 10 is locked to more reliably keep the closed state.

In FIG. 6, the lock includes a perforated lock band 43 and a hook 44. The lock band 43 as a lock and the band 41 as an attaching tool are strongly bonded to the lower surface of the case 10 (the outer surface of the bottom surface of the lower half body 21) in an overlapping manner in this order. The band 41 is loosely attached to the wrist of the patient as described above, but the locking band 43 is unreleasably fastened with hook 44 such that both end portions wind the case 10 tightly from opposite to each other (preferably, both end portions are less than one turn). Since the case 10 is tightly wound by the lock band 43, the upper and lower half bodies 11 and 21 cannot be opened unless the band 43 is cut with scissors or the like. The lock band and the hook are included in the concept of a lock in this specification in the sense that the case is not physically opened rather than sealed.

FIG. 7 illustrates a padlock. Hook rings 45 and 46 are respectively fixed to side surfaces (side wall) of the upper and lower half bodies 11 and 21 of the case 10 on the side opposite to the hinge 31. A hook of a lock 47 is passed through these hook rings to lock. The case 10 cannot be opened unless a key is used.

Although a padlock is drawn in FIG. 7 for easy understanding, it is needless to say that the lock may be incorporated (built-in lock) over the upper and lower half bodies 11 and 21 of the case 10. Instead of providing the removal-preventing column 14 fixedly in the lower half body 21, a central shaft or a receiving cylinder may be fixedly provided in the lower half body 21 and used to rotatably support a removal-preventing column, and a mechanism (e.g. rachet mechanism) may be provided to prevent rotation of the removal-preventing column at least one direction to constitute a resistance member.

### Second Embodiment

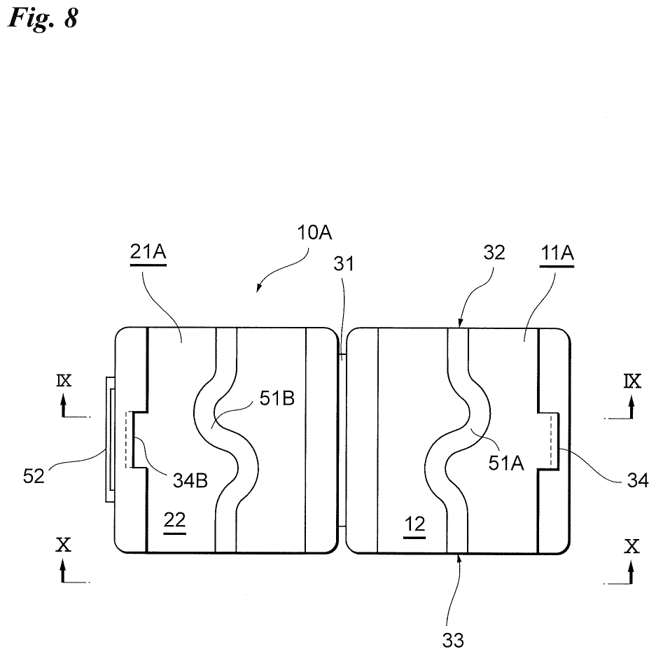

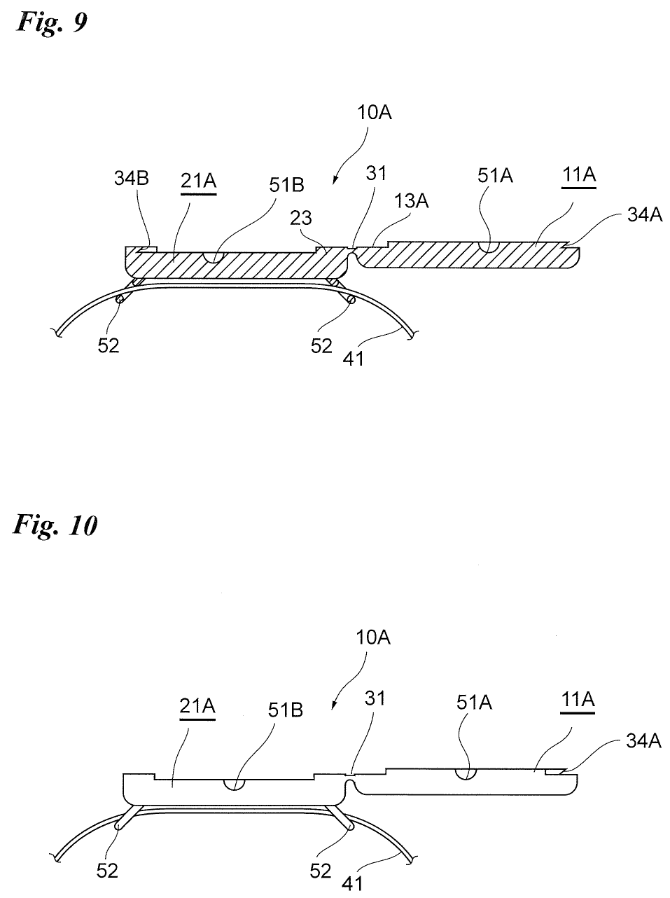

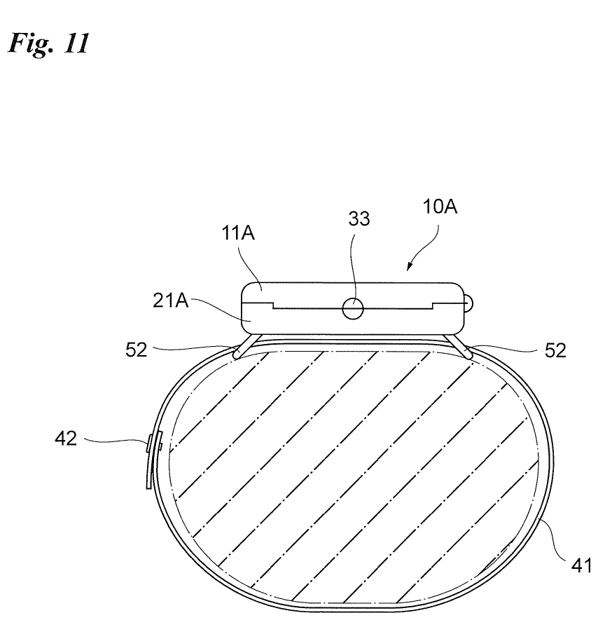

FIGS. 8 to 11 illustrate a second embodiment. FIG. 8 illustrates the inside of the case by opening the upper and lower half bodies of the case, FIG. 9 is a cross-sectional view taken along line IX-IX of FIG. 8, FIG. 10 is a view as seen from line X-X of FIG. 8, and FIG. 11 illustrates a state in which the case is attached to the wrist in a cross section of the wrist. The members corresponding to the members in the first embodiment are denoted by the same reference numerals as those in the first embodiment, and redundant description will be avoided.

In the second embodiment, removal-preventing or retaining grooves (resistance members) 51A and 51B are formed on the inner surfaces of the upper and lower half bodies 11A and 21A, respectively. The removal-preventing grooves 51A and 51B are meandering (meandering with a curve) grooves, and when the upper and lower half bodies 11A and 21A are closed and their inner surfaces are joined, the removal-preventing grooves 51A and 51B overlap each other in a coincident manner, and become meandering holes (removal-preventing or retaining holes). The inner diameter of this hole is substantially the same as the outer diameter of the tube. Although the cross sections of these grooves 51A and 51B are both semicircular, the cross section of one groove may be slightly closer to a circular shape than the semicircular shape, and the cross section of the other groove may have a shape in which a part of the semicircular shape is missing. The portions of the upper and lower half bodies 11A and 21A where the removal-preventing grooves 51A and 51B are formed may be referred to as resistance members.

In order to form the removal-preventing grooves 51A and 51B, the upper and lower half bodies 11A and 21A are slightly thick, and the lower half body 21A has the frame portion 23, but the corresponding periphery of the upper half body 11A is absent by the frame portion 23 (indicated by reference numeral 13A). In addition, in the lower half body 21A, band passageways 52 are formed on the lower surfaces of both sides to face obliquely laterally. In this embodiment, the resistance half ring body is not provided at a corresponding portion of the inlet and the outlet (hole). Needless to say, a resistive half ring body may be provided. The tube can be tightly fixed when the inner diameter of the holes of the inlet and outlet (holes) of the case 10A is slightly smaller than the outer diameter of the tube. The inner diameter of the inlet and outlet of the case 10A may fix the tube to the extent that the tube is prevented from loosening in the case.

In this embodiment, in a state where the upper and lower half bodies 11A and 21A of the case 10A are opened, the drip tube 2 is partially fitted along the removal-preventing grooves 51B or 51A of the lower half body 21A or the upper half body 11A, and then the upper and lower half bodies 11A and 21A are closed and coupled by the engaging portion 34. Further, the lock is firmly locked by the lock band 43.

Since the tube 2 is accommodated in the removal-preventing hole formed by the meandering grooves 51A and 51B, resistance against tensile force is strong. Regardless of intention or carelessness, the tube 2 is extremely difficult to come off from the case 10A. The tube 2 is prevented from being extracted or removed. The perforated band 41 for attachment passes through the band passage 52 and is attached to the wrist by the hook 42. In the present embodiment, the wearing feeling of the case 10A is similar to the wearing feeling of a wristwatch, and there is no or little discomfort. Since the tube 2 only passes through the case 10A, the thickness of the case 10A can be further reduced.

### Third Embodiment

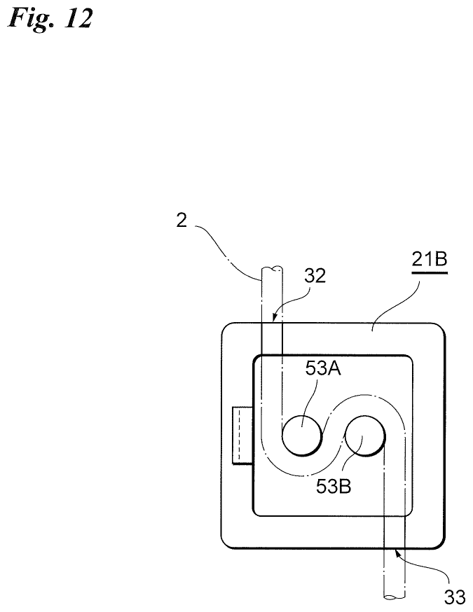

The third embodiment shows various configurations of the resistance members for preventing removal and retaining tube provided on the case. An example thereof is illustrated in FIGS. 12, 13, and 14. In these drawings, only the inner surfaces of lower half bodies 21B, 21C, and 21D are shown.

In FIG. 12, two removal-preventing columns 53A and 53B are erected on the lower half body 21B. An inlet (hole) 32 formed on one end surface (wall) (side surface) of the case is located on the left side in the drawing, and an outlet (hole) 33 formed on the other end surface (side surface) of the case is located on the right side. In this manner, on the opposing ends of the case, the inlet and the outlet do not necessarily have to be provided at the center matching positions as illustrated in FIGS. 2 and 8, and the inlet and outlet may be located at positions shifted to the left and right. The tube 2 that has entered the case from the inlet 32 travels straight, is first hooked on the first column 53A, is changed in the opposite direction and hooked on the second column 53B, and is changed in the direction to reach the outlet 33 and exit.

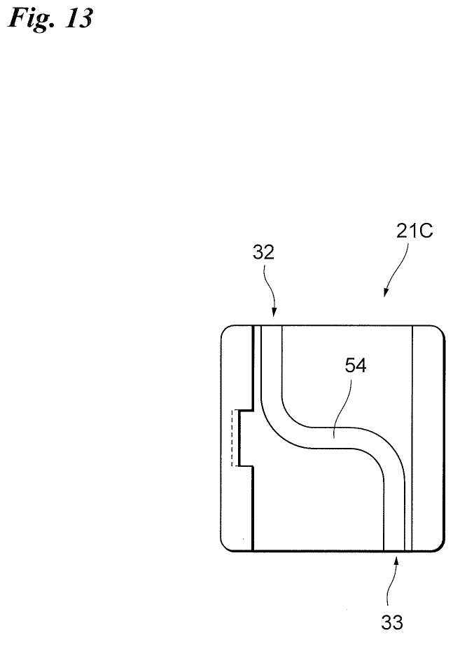

In FIG. 13, a removal-preventing or retaining groove (resistance member) 54 goes straight from the inlet 32 formed on the left side of the case, curves in a substantially right angle direction, further curves in a right angle direction, and goes toward an outlet 33. The tube is housed along this groove 54. When the upper half body is closed, grooves of a matching shape are formed, and a passage (a removal-preventing hole) of the tube is formed by these grooves.

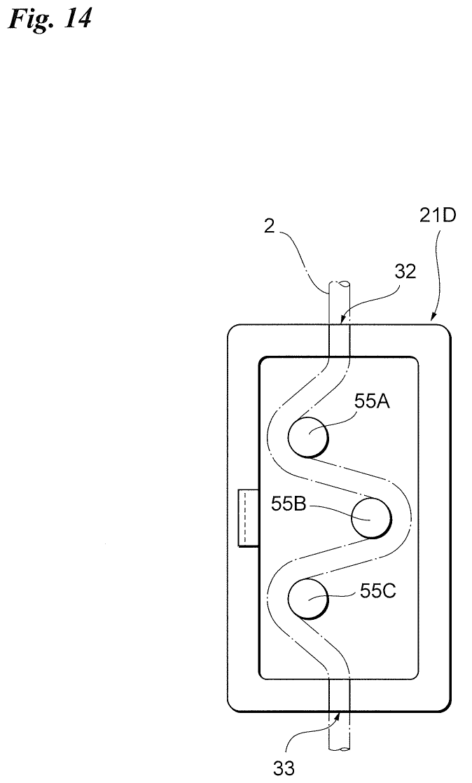

In FIG. 14, the case is slightly long in the direction connecting the inlet and outlet, and three removal-preventing or retaining columns (resistance members) are provided. These columns 55A, 55B, and 55C are erected in the lower half body 21D while being slightly laterally alternately shifted from the straight line connecting the inlet and the outlet. The tube 2 that has entered the case from the inlet 32 comes into contact with the left side of the column 55A, then meanders to come into contact with the right side of the column 55B, and further bends to come into contact with the left side of the column 55C and exits the case from the outlet 33. When a tensile force acts on the tube, the tube 2 comes into pressure contact with these columns to resist the tensile force.

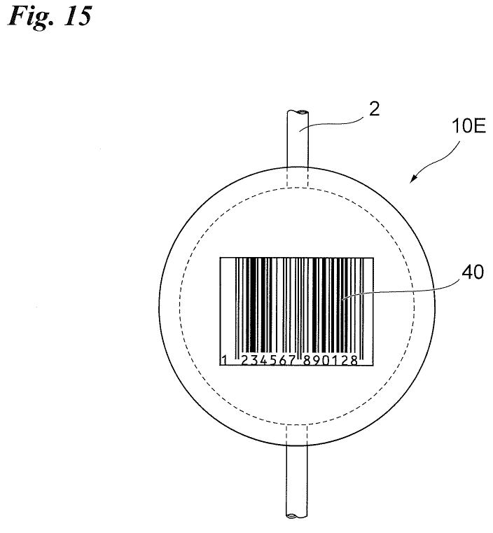

FIG. 15 illustrates a modified embodiment of the case. In the embodiments described above, the case has a square shape when viewed from the upper surface, but in FIG. 15, a case 10E has a circular shape when viewed from the upper surface. In such a round case, it is preferable that the upper half body is not coupled to the lower half body with a hinge, but is separable from the lower half body, and is coupled to the lower half body by fitting, screwing, or a combination of screwing (preferably having a small rotation angle) and engagement. Of course, coupling by a hinge and an engaging portion may be used. The resistance member provided in the case may be either a column or a groove. Furthermore, various shapes such as an elliptical shape can be adopted as the case. In these circular, elliptical, and the like cases, the side surface (wall) on which the inlet and the outlet are provided is a peripheral surface (wall), and the opposing position is not necessarily in the direction opposite by 180 degrees. When a band passer for a perforated band for locking is provided on the upper surface of the case, and the band is fastened by a hook after being passed through the band passer, the band does not slip off. Adding decorations or making it colorful will reduce the patient's resistance.

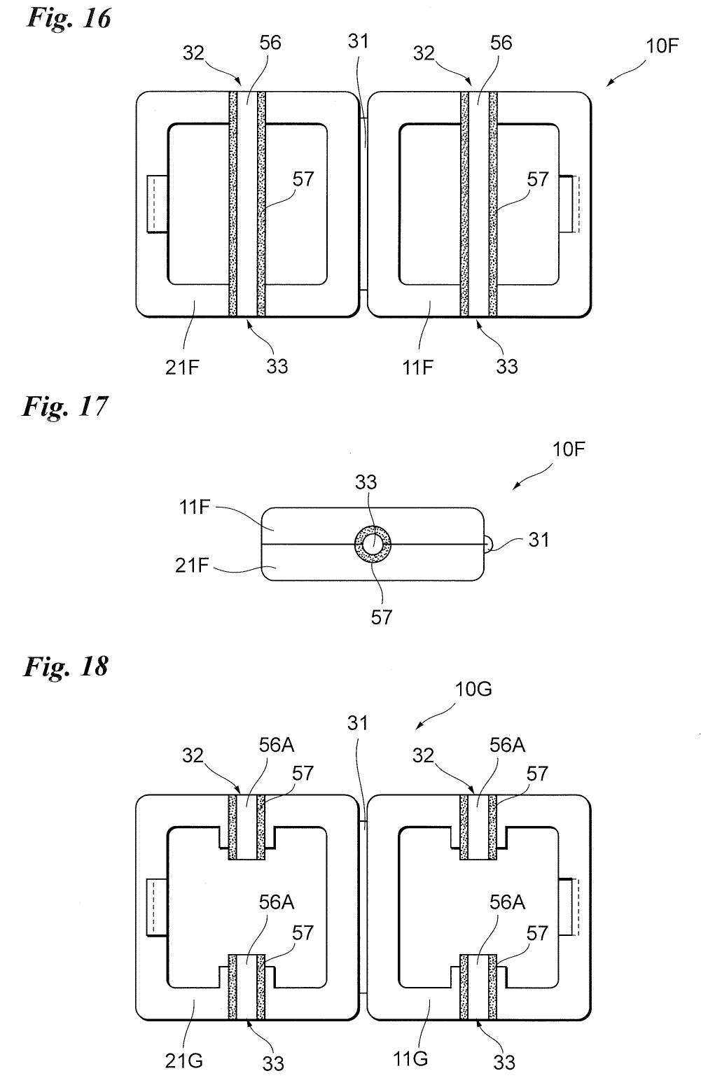

FIGS. 16 to 18 illustrate the use of a resistance semi-cylindrical body having a large slip resistance, and each of the removal-preventing or retaining grooves is linear.

In FIGS. 16 and 17, a groove 56 having a semicircular cross section is formed between the inlet and the outlet of the upper and lower half bodies 11F and 21F respectively, and a semi-cylindrical resistive half cylinder 57 having a large sliding resistance (semicircular cross section) is fitted to the inner surface of the groove 56 and fixed to the groove 56.

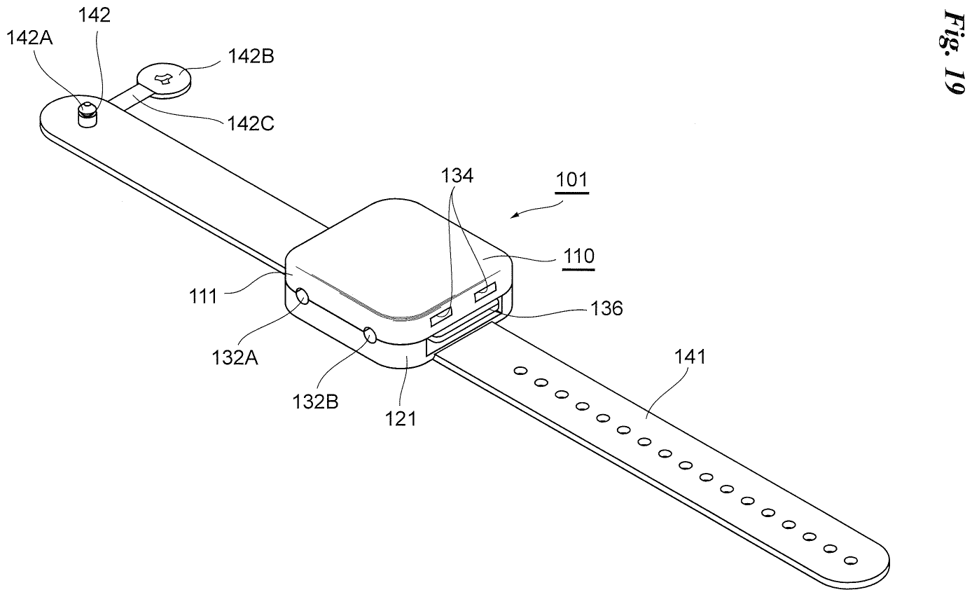

In FIG. 18, in the upper and lower half bodies 11G and 21G respectively, a groove 56A and a resistance half cylinder 57 are provided only in a portion corresponding to an inlet and an outlet (hole). A removal-preventing column may be provided in a space in a central portion of the upper and lower half bodies 11G and 21G. In addition, the upper half body may be made transparent or may have a lattice shape or a mesh shape so that the inside of the case can be seen. Furthermore, the width of the frame portion in the case may be increased, and the removal-preventing groove (and the resistance half cylinder) may be formed only within the range of the width of the frame portion having the inlet and the outlet (hole) (also used as inlet and outlet (hole)). The inlet, the outlet, and the removal-preventing groove may be inclined rather than perpendicular to the side surface (end surface) of the case. The inlet and outlet may be provided with a semi-inner tubular portion (tubular portion) protruding outward. All of these correspond to providing a resistance member in the case. It goes without saying that the lock of the case may also be provided in the second embodiment, the third embodiment, and a fourth embodiment described later, or the features of the first, second, third, fourth embodiments, and modified embodiments may be combined.

### Fourth Embodiment

There are mainly two types of tube anti-extraction devices according to the fourth embodiment, which includes a device in which the case has two holes and a device in which the case has four holes. A type having three or five or more holes and a type having one hole are described at the end.

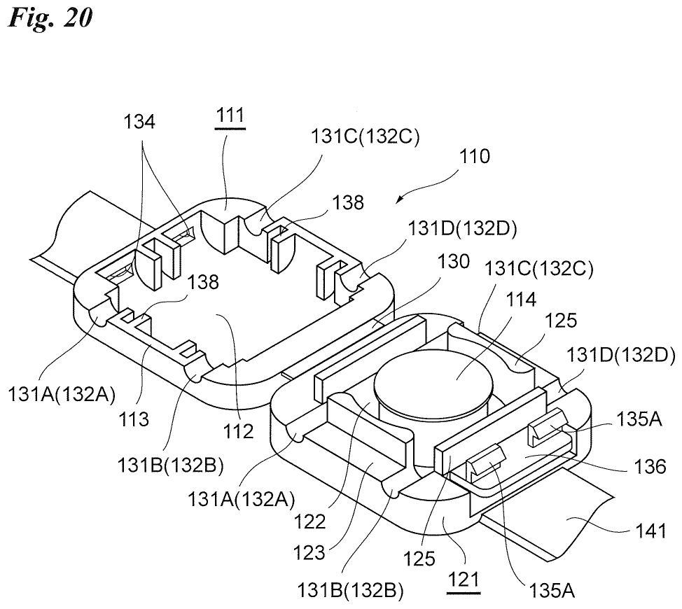

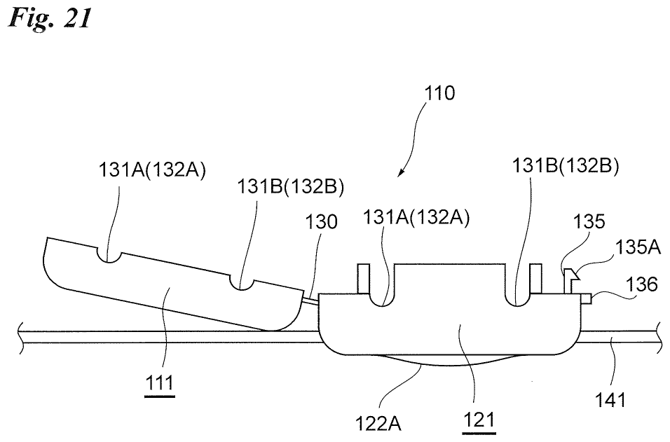

First, a device according to an embodiment in which four holes are provided in a case will be described. FIG. 19 illustrates the entire tube anti-extraction device. FIG. 20 illustrates the inside of the upper and lower half bodies of the case by opening the upper and lower half bodies. FIG. 21 is a side view of the opened case. The case has a thin rectangular parallelepiped shape (rounded at the corner) in a state where the upper and lower half bodies described later are closed.

As in the first embodiment, a tube anti-extraction device 101 includes a case 110 and a perforated band (attaching tool) 141 for loosely attaching the case 110 to a wrist, an ankle, or the like of a person. The case 110 is formed of a hard plastic, and includes an upper half body 111 and a lower half body 121 openably coupled by a hinge 130. The lower half body 121 is in contact with human skin. On a bottom surface (plate) 122 of the lower half body 121, the perforated band 141 is sandwiched and fixed between the double bottom surface (plate) members. The outer surface of the bottom surface 122 of the lower half body 121 in contact with the human skin is gently curved in a convex shape (indicated by reference numeral 122A), prevents sweating, and provides a comfortable wearing feeling. A hook 142 of the band 141 has a protrusion 142A provided at the end of the band and a portion 142B connected to the same end of the band by a connecting portion 142C and having a hole.

The upper half body 111 has a bottom surface (plate) (corresponding to the upper surface of the case) 112 and a frame portion 113 around the bottom surface. Similarly, the lower half body 121 has a bottom surface (plate) 122 and a frame portion 123 around the bottom surface. Portions 131A, 131B, 131C, and 131D that are absent (lacking) (recess) in a semicircular shape are formed in portions of the frame portions 113 and 123 of the upper and lower half bodies 111 and 121 parallel to the extending direction of the band 141, and these semicircular shape absent portions 131A, 131B, 131C, and 131D form circular holes 132A, 132B, 132C, and 132D when the upper and lower half bodies 111 and 121 are closed (FIG. 19 illustrates only holes 132A and 132B). These holes are formed in a total of four by two on opposing side surfaces of the case (side surfaces parallel to the longitudinal direction of the band), and are used as an inlet or an outlet for the tube as will be understood from the following description.

The hinge 130 that openably connects the upper and lower half bodies 111 and 121 is provided integrally with the case on a side surface orthogonal to the side surface on which the holes are formed. In the upper half body 111, two engagement windows 134 are formed in the frame portion 113 on the side opposite to the side on which the hinge 130 is provided. On the inner surface of the upper half body 111, a reinforcement rib 138 is provided at an appropriate position.

Inside the lower half body 121, at the center, a removal-preventing or retaining column (resistance member) 114 is fixedly provided on the bottom surface (plate) 122. Around the removal-preventing column 114, tube guides 125 are fixedly provided at four locations away from each other along the inner surface of the frame portion 123. The inner surface of the tube guide 125 that is parallel to the band 141 is recessed in an arc shape to guide the tube wound around the removal-preventing column 114. In the inner surface of the bottom surface 122, the periphery of the removal-preventing column 114 is a groove deeper than the upper surface of the frame portion 123 in order to pass the tube. The linear groove in the direction connecting the holes 132A and 132C and the holes 132B and 132D is deeper.

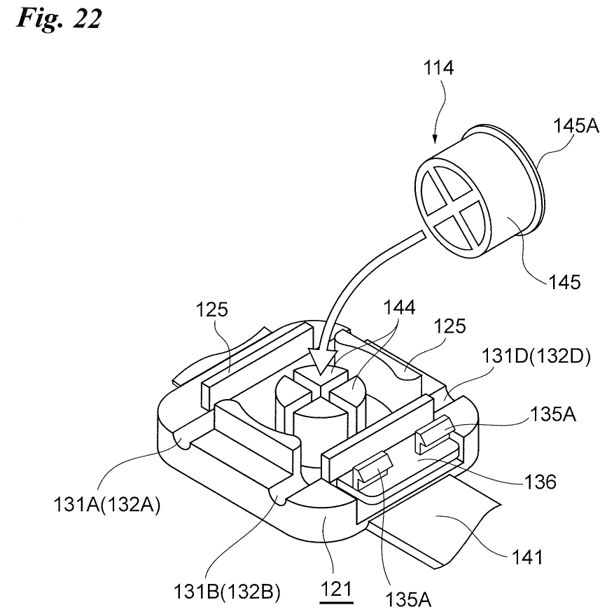

As illustrated in FIG. 22, the removal-preventing column 114 includes a receiving portion 144 made of four small columns fixed to the inner surface (plate) of the lower half body 121, and a cylindrical crown 145 made of soft rubber and placed over the receiving portion 144. Holes into which the small columns of the receiving portion 144 enter are formed inside the crown 145. The crown 145 is further formed with a flange 145A slightly protruding in the outer peripheral direction from the upper surface thereof. The flange 145A prevents the tube wound around the removal-preventing column 114 from jumping up due to its elasticity. Since the crown 145 is formed of soft rubber, the frictional force with respect to the tube wound around the crown is high.

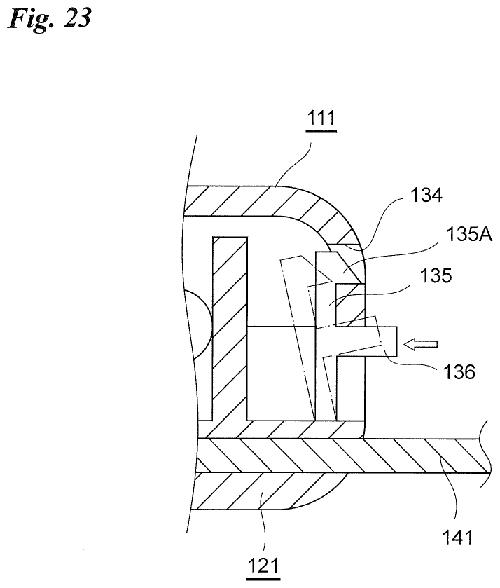

Referring to FIG. 23, in the lower half body 121, an engagement member 135 having two engagement claws 135A at the upper end is further erected and fixed in the side portion on the opposite side of the hinge 130. When the upper and lower half bodies 111 and 121 are closed, these engagement claws 135A engage with the engagement windows 134 of the upper half body 111 to keep the upper and lower half bodies 111 and 121 closed. The engagement window 134 and the engagement member 135 (engagement claw 135A) constitute a fastener. A releasing pressing portion 136 is provided integrally with the engagement member 135 on the outer surface of the engagement member 135. The outer end of the releasing pressing portion 136 slightly protrudes outward from the case 110. By pushing this protruding outer end with a fingertip, the upper end of the engagement member 135 is warped inward as indicated by a dot-and-dash line in FIG. 23, and the engagement between the engagement claw 135A and the engagement window 134 is released. As a result, the upper and lower half bodies 111 and 121 are opened.

The engagement between the engagement claw 135A and the engagement window 134 is released by pressing the releasing pressing portion 136 with a finger. As described above, the engagement device that can be released by directly operating with a hand or a finger without using a jig (tool) is a fastener. Of course, if the elasticity of the engagement member 135 is increased, it cannot be released with one hand (a patient wearing the present anti-extraction device in one hand can use only the other hand or finger), and thus it is close to a lock, but a nurse can release it without using a jig. In the case of a structure in which the releasing pressing portion 136 is accommodated in the case without being taken out of the case, a jig for pressing the pressing portion 136 from the outside is required, and thus, in this case, the lock is used. This is because the jig corresponds to a key.

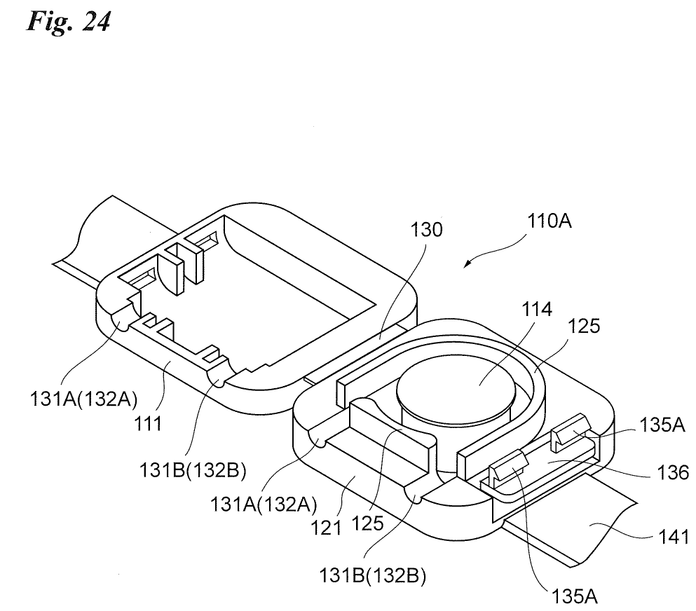

Next, a modified embodiment in which two holes are provided in the case will be described with reference to FIG. 24. As can be seen from comparison between FIGS. 24 and 20, in this modified embodiment, only semicircular absent portions 131A and 131B (holes 132A and 132B) are provided in the upper and lower half bodies on one side of the case 110A. That is, the case 110A of the anti-extraction device 101A has two holes.

The case 110A of this modified embodiment is provided with two holes. In the lower half body 121, a groove through which a tube passes is formed around the removal-preventing column 114, and is connected to the recess portions 131A and 131B. The tube guide 125 is provided around the groove in the lower half body 121. Other configurations, that is, the upper and lower half bodies are openably connected by the hinge 130, and configurations of the removal-preventing column 114, the fastener, and the like are the same as those of the embodiment having four holes. In the case of two holes, there are a case having the hole 132A on one side and a hole 132C on the other side, and a case having the holes 132B and 132D.

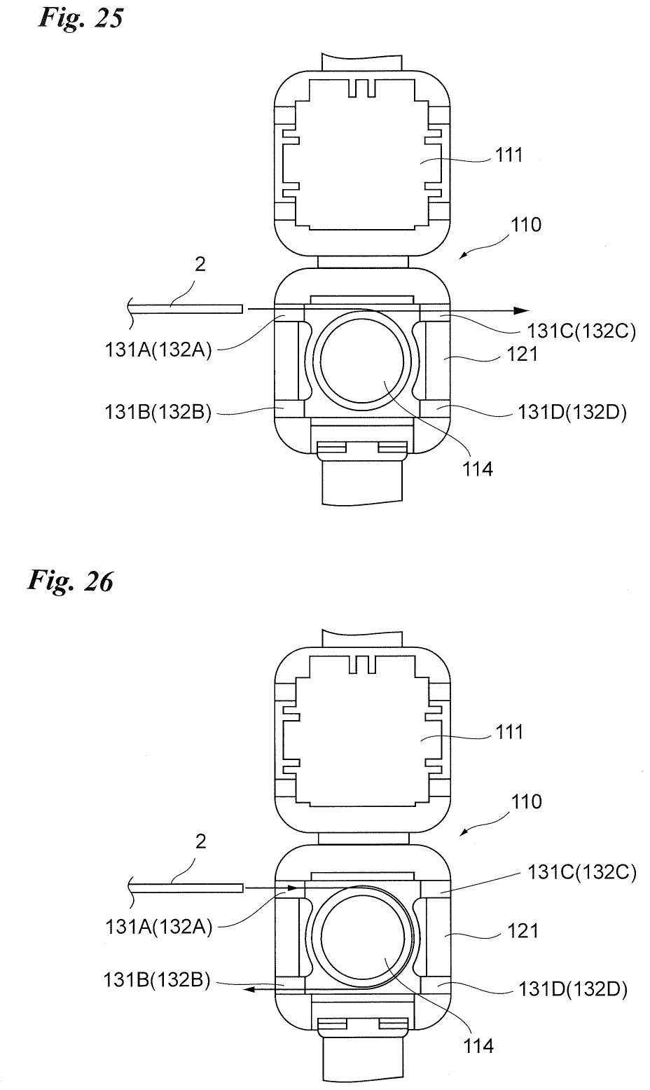

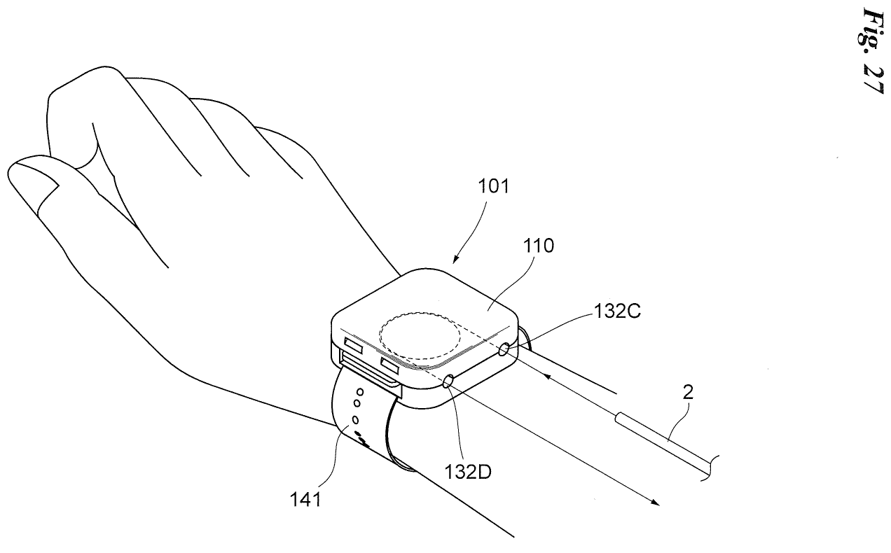

A using method of these anti-extraction devices 101 and 101A will be described with reference to FIGS. 25 and 26. In these drawings, the device 101 (case 110) having four holes is illustrated.

In FIG. 25, the tube 2 enters the case 110 from the circular hole 132A of the case 110, goes around the removal-preventing column 114 one turn, and is drawn out from the hole 132C to the outside. Even if the tube protruding to the outside is pulled in the protruding direction, the tube is retained and prevented from being extracted by the frictional force between the tube and the removal-preventing column 114.

In FIG. 26, the tube 2 enters through the circular hole 132A in the case 110, makes one and a half revolutions around the removal-preventing column 114, and then protrudes to the outside through the hole 132B. Also in this case, even if the portion of the tube 2 that has come out of the case is extracted, the tube is prevented from coming off.

Similarly, the tube can be prevented from coming off by using a combination of other holes 132B and 132C or a combination of holes 132B and 132D. In the case of using the device 101A having two holes, it is possible to use one and a half turns of FIG. 26.

There is also a modified embodiment in which only one hole is provided in the case. In this case, the hole is formed slightly long in the vertical direction, the tube is introduced into the case from one hole, and the tube is wound around the removal-preventing column one and a half times to be led out from the same hole. The tube portion introduced into the case and the tube portion led out from the case vertically overlap each other.

Similarly, there is also a modified embodiment in which three or five or more holes are provided in the case. In this case, in particular, there may be a circular case when viewed from a plane as illustrated in FIG. 15. In the case of a circular case of three holes, as an embodiment, one hole may be provided within an angle range obtained by dividing 360° into three, and in the case of five holes, one hole may be provided within an angle range obtained by dividing 360° into five. Of course, it is not limited to the three equal parts and the five equal parts, and one hole may be provided in an appropriate angle range.

FIG. 27 illustrates a using method different from that in FIG. 1. The tube 2 connected to the IV needle punctured in the forearm is introduced into one hole 132C of the case 110 having four holes attached to the wrist by the band 141, wound one and a half turns inside the case 110, extracted to the outside of the case 110 from another hole 132D on the same side as the hole into which the tube is introduced, and guided in the direction of the puncture site. It is extremely advantageous since the direction of guiding the tube from the case 110 can be arranged in a desired direction in consideration of the arrangement of the drip orthosis, the posture of the patient, the position of the nurse, and other circumstances.

The above embodiments prevent extraction of a tube connected to an IV needle (catheter), but as described above, there are a blood transfusion tube (conceptually included in the drip tube), a peripherally inserted central catheter (PICC) line, a hemodialysis tube, and the like in addition to the drip tube, and the puncture sites (areas) are different from each other. In any case, if there is a puncture site in the left or right upper limb, the anti-extraction device is correspondingly attached to the left or right wrist, and if there is a puncture site in the left or right lower limb, the anti-extraction device is correspondingly attached to the left or right ankle.

What is claimed is:

1. A tube anti-extraction device comprising:
a case having a sidewall formed with a hole for passing a tube therethrough; a resistance member provided in the case for resisting a tensile force acting from outside the case on the tube passing out of the hole; and a tool for attaching the case to a wrist on an upper limb or an ankle on a lower limb where a puncture site is present.

2. The tube anti-extraction device according to claim 1, wherein two holes passing the tubes are formed on a side wall of the case.

3. The tube anti-extraction device according to claim 1, wherein four holes passing the tubes are formed on a side wall of the case.

4. The tube anti-extraction device according to claim 1, wherein the resistance member is all or a part of a removal-preventing or retaining column around which the tube is wound and a curved or linear groove or hole for preventing removal of or retaining along which the tube is running in the case.

5. The tube anti-extraction device according to claim 4, wherein the removal-preventing or retaining column is formed of a soft member.

6. The tube anti-extraction device according to claim 4, wherein the removal-preventing or retaining column is provided with a flange extending outward at an upper end portion thereof.

7. The tube anti-extraction device according to claim 5, wherein the removal-preventing or retaining column includes a soft member crown that covers a receiving portion in the case.

8. The tube anti-extraction device according to claim 1, wherein the case includes at least two partial bodies, and these partial bodies are detachably coupled by any one or a combination of a hinge, engagement, fitting, and screwing.

9. The tube anti-extraction device according to claim 8, wherein absent portions for hole formation are formed in each of the at least two partial bodies, and when the partial bodies are combined, the absent portions are met to form the hole.

10. The tube anti-extraction device according to claim 8, further comprising: a fastener that keeps at least two partial bodies forming the case coupled.

11. The tube anti-extraction device according to claim 8, further comprising: a lock that keeps at least two partial bodies forming the case coupled.

12. A case for a tube anti-extraction device, the case comprising:
a case body having a sidewall formed with a hole for passing a tube therethrough and a resistance member provided within the case body for resisting a tensile force acting from outside the case body on the tube passing out of the hole.
